(19) United States
(12) Patent Application Publication
(10) Pub. No.: US 2010/0204727 A1
(43) Pub. Date: Aug. 12, 2010

(54) Magnetic Surgical Device to Manipulate Tissue in Laparoscopic Surgeries or via Natural Holes Performed with a Single Trocar

(76) Inventor: Guillermo Manuel Dominguez, Buenos Aires (AR)

Correspondence Address: CROWELL & MORING LLP;INTELLECTUAL PROPERTY GROUP, P.O. BOX 14300, WASHINGTON, DC, 20044-4300, US

(21) Appl. No.: 12/700,348

(22) Filed: Feb. 4, 2010

Related U.S. Application Data
(63) Continuation of application No. PCT/EP2008/060338, filed on Aug. 6, 2008.
Application No. PCT/EP2008/060338 is a continuation-in-part of application No. 11/834,746, filed on Aug. 7, 2007.

(51) Int. Cl. A61B 17/28

(52) U.S. Cl. 606/205

(57) ABSTRACT

The present invention is related to surgical instruments incorporating the usage of magnets for application in minimally invasive surgery to perform abdominal surgery with a single incision through the navel which is the most widely used, this incision can also be done through some natural hole like the vagina, mouth, etc. Generally, the invention comprises instruments to perform cholecystectomy (gallbladder removal), but they are also useful for all type of operation requiring mobilization, traction, counter-traction or also separation of abdominal organs. They can be used in several kinds of surgeries, such as laparoscopy, general, gynecological, and urologic surgery.

This application is a continuation of international PCT application PCT/EP2008/060338, filed Aug. 6, 2008, the entire disclosure of which is expressly incorporated herein by reference, and is a continuation-in-part of, and claims priority of, prior U.S. patent application Ser. No. 11/834,746, filed Aug. 7, 2007, the entire disclosure of which is also expressly incorporated herein by reference.

### FIELD OF THE INVENTION

The present invention is related to surgical instruments that incorporate the use of magnets for application in minimally invasive surgery to perform abdominal surgery with a single incision usually done through the navel; or else this incision can also be done through some natural hole in human body such as the vagina, mouth or anus.

The present invention also describes surgical tools to manipulate the magnetic surgical devices, a surgical apparatus to position an external magnet during a surgery, a surgical probe that comprises at least one magnet in one of its ends, a surgical tool to manipulate spherical magnets, a catheter cannula with a system to fasten a preformed knot, and a surgical organ retractor.

Generally the invention comprises instruments to perform cholecystectomy (gallbladder removal), but may also be used for all type of surgical procedure requiring mobilization, traction, counter traction or also abdominal organs separation. They can be used in laparoscopic, gynecologic, urologic, pediatric surgeries.

### BACKGROUND OF THE INVENTION

In 1997 Dr. Fausto Davila Avila, in Mexico, invented a surgical technique which he called “no track” surgery that featured a single trocar usage, plus an optic with working canal aided by 1 to 1.2 mm diameter percutaneous needles and submitted his paper to the Society of American Gastrointestinal Endoscopic Surgeons in 2004. This work is described in “Substitution of ports by percutaneous needles in endoscopic surgery” (“Sustitución de puertos por agujas percutáneas en cirugía endoscópica”). Rev. Mex. Cir. Endoscop. 2004; 5:172-178, Dávila F, Sandoval R, Montero Pérez J, Dávila Or, Davila M, Alonso J, Lemus J.

In that time, the usage of magnets started for several medical specialties. In Gatroeneterology: since December 2001 Villaverde A., Cope C at al. made compression gastroenteroanastomosis with magnets inserted via endoscopic or fluoroscopic means.

Related publications are: Creation of compression gastroenterostomy by means of oral, percutaneous, or surgical introduction of magnets: feasibility study in swine. Cope C. JVIR 1995; 6:539-545; Evaluation of compression cholecystogastric and cholecystojejunal anastomoses in swine after peroral and surgical introduction of magnets. Cope C. JVIR 1995; 6:546-552. Stent placement of gastroenteric anastomoses formed by magnetic compression. Cope C; Clark T; Ginsberg G; Habecker P. JVIR 1999; 10:1379-1386. Long-term patency of experimental magnetic compression gastroenteric anastomoses achieved with covered stents. Cope C; Ginsberg G. Gastrointestinal Endoscopy June 2001 Volume 53 Number 7. Magnet usage for valves adjustments used in hydrocefalic patients. Neurocirugía. 55 (3): 627-630, September 2004. Jandial, Rahul M. D.; Aryan, Henry E. M. D.; Hughes, Samuel A. M. D., Ph.D.; Collection, Michael L. M. D., Ph.D.

Magnets are used on animals in robotic surgery. Dr. H. Rivas published: Magnetic positioning system to drive trocarles laparoscopic instruments. Dr Dimitri Oleynikov published: An in vivo mobile robot for surgical vision and task assistance, University of Nebraska.

U.S. Pat. No. 5,690,656 describes carrying out an anastomosis between two hollow viscera using magnets. Described are a method and a device to perform abdominal viscera anastomoses. To this end, a first magnet is set in place inside the first viscera, and a larger magnet inside the second viscera. The magnets attract each other so gripping a portion of the visceral walls between them. The method to perform an anastomosis of viscera comprises the following steps: (a) inserting a magnet in the viscera adjacent to a digestive zone; (b) inserting a second magnet in the stomach, thereby the second magnet is brought to the digestive zone until it attracts the first magnet and it is brought together in addition through a wall of the digestive zone and a visceral wall; and (c) causing the fine tissue trapped between magnets to undergo an ischemic necrosis.

U.S. Patent Application 2003/0114731 describes a method and apparatus to perform a surgery using a magnetic field. One or more magnets are used in the surgery arranged outside the patient body and they are used to position, to guide and to anchor surgical instruments and/or camera located inside the patient during surgery. The system is called MAGS (Magnetic anchoring and guidance system).

### SUMMARY OF THE INVENTION

The invention consists of using very high magnetic intensity magnets, preferably made of a rare earth, for example neodymium, but alternatively also with other magnetizable materials, inside and outside the abdominal cavity, in different forms and sizes, and plated with as gold, silver, or nickel, or any other materials being biocompatible with usage in human body, along with specially designed instruments to be used with magnets in laparoscopic surgery. The material going along with the magnets may be a magnetizable medical grade steel or alternatively a non-ferromagnetic medical grade steel, that is a non magnetizable medical grade steel, or else a biocompatible medical grade plastic. The usage of a non magnetizable material will avoid the tooling from interacting with the magnetic field and, at the same time, will avoid affecting the tissues when acting as a separator. Non ferromagnetic steel is known in the art and is used in several industrial activities. For example, steels containing low carbon percentages, for example SAE 304 or 316L steel. However, also steel with low contents of manganese and chrome such as the one revealed in U.S. Pat. No. 4,481,033 is non magnetic and also shows good corrosion resistance. By means of magnets and the magnetic field generated through the abdominal wall it is possible to move, push, pull, separate and elevate the organ or organs in order to carry out navel surgery through a single 11 mm incision. Although the navel is the most frequently used access spot in laparoscopy, it is also possible to make this single incision with flexible surgical endoscopes entering the abdominal cavity by the vagina using optics with built-in working canal which allow use of surgery instruments. Different types of surgeries may be performed taking advantage of the magnetic field generated by an external magnet and a magnet placed inside an abdominal cavity organ, plus the instruments entering the same hole where the optics enters.

Magnets are integral part of specially designed instruments to be used in laparoscopic or mini-invasive surgery. As was already mentioned before, preferably, the material going along with the magnets may be mangetizable, but should preferably be of a non magnetic steel or medical grade plastic, in order to help keeping a gap between the magnet and the organ and also for constructing complementary tooling for helping in the surgery process.

The device comprising the magnets enters the abdominal cavity by an umbilical trocar, it grabs and internal tissue, and consequently is able of moving, pulling and pushing the organs. To achieve this, the magnets fastened to the organ are manipulated by means of clips, and are further attracted and moved along by external magnets. Also, magnets to be used in a near future will be insertable through the human natural holes like mouth, anus, and vagina. They can also be placed guided by radioscopy or endoscopy in the site of interest like the stomach, colon sigmoides, etc.

It is also possible to push lithos from the biliary tract to the duodenum by type k108 probes with internal or spherical magnets of neodymium or ferromagnetic small steel balls placed via transcystic or transcholedochus, and pulled by external magnet for moving within the biliary tract, guided by radioscopy. In female patients a 12-15 mm trocar could be used, inserted via vagina (bottom of Douglas' sac) for performing all the surgery process through that hole or, for example, using mechanical sutures or optionally extracting surgical parts.

This technique with magnets also can be applied in traditional laparoscopic surgery, robotic surgery, surgery through natural holes like accessory instruments of transgastrig, transvaginal surgery, etc.

These objects will be apparent for the experts in the art by the following description.

### DESCRIPTION OF THE FIGURES

FIG. 1 shows a magnetic surgical device for manipulating tissue during a so called Dominguez tandem surgery according to a first object of the present invention.

FIG. 2 shows a layout of the clip for the anchoring of organs subject of the present invention.

FIG. 3 shows the organ anchoring clip fully assembled which is one of the objets of the present invention.

FIG. 4 shows in detail the plurality of annular threaded magnets which are integral part of the set of FIG. 1.

FIG. 5 shows a surgical tool called Thomas pliers used for manipulating a clip such as the one shown in FIGS. 2 and 3 according to a second object of the present invention.

FIG. 6 shows in detail the anchoring end of the so called Thomas pliers shown in FIG. 5.

FIG. 7 shows the so called Thomas pliers shown in FIG. 5 when used anchoring a clip from behind.

FIG. 8 shows the so called Thomas pliers shown in FIG. 5 when used perpendicularly anchoring a clip.

FIG. 9 shows an external surgical magnet used to manipulate a magnetic surgical device as the one shown in FIG. 1, inside a human body during surgery.

FIG. 10 shows a mechanical apparatus rotatable up to 360°, for manipulating an external surgical magnet like the one shown in FIG. 9 during surgery.

FIG. 11 shows a probe comprising a plurality of magnets inside one of its ends, which is used together with a magnetic surgical device as the one shown in FIG. 1 for manipulating hollow organs.

FIG. 12 shows a practical use of probes shown in FIG. 11.

FIG. 13 shows a surgical tool for manipulating spherical magnets or magnets with central hole, called Camila pliers.

FIG. 14 shows a cannula with a system for fastening knots called Valentina cannula which is used to take cholangiographies.

FIG. 15 shows in detail the end of the Valentina cannula as the one shown in FIG. 14, which is inserted into the patient.

FIG. 16 shows an organ separator or retractor called William separator or retractor.

FIG. 17A shows another type of crocodile clip, FIG. 17B shows a perspective view of the crocodile clip of FIGS. 2 and 3, and FIG. 17C shows other possible embodiments of fixing clips.

FIGS. 18A and 18B show the tube made of ferromagnetic material which may act as a housing of one or more magnets, linked to the crocodile clip. FIG. 18C shows the tube with the clip and a security string.

FIG. 19 shows a lengthwise cut of the tube shown in FIGS. 18A-18C, which acts as a housing of the one or more magnets.

FIG. 20 shows a perspective view of back opening of the tube shown in FIGS. 18A-18C.

FIG. 21 shows a perspective view of the pot-shaped support of FIG. 9, which allows regulating the external magnet's distance to the patient's skin, with the regulating pin completely raised.

FIG. 22 shows a perspective view of the pot-shaped support of FIG. 9 which allows regulating the external magnet's distance to the patient's skin, with the regulating pin completely lowered.

FIG. 23 shows a perspective view of the base hole of the pot-shaped support of FIGS. 21 and 22, and its inner part.

FIG. 24 shows a perspective view of the pot-shaped support of FIGS. 21-23 with a different mounting of the regulating pin.

FIG. 25 shows another embodiment of a hepatic separator device.

FIG. 26 shows an assembly formed by the clip of FIG. 3 linked to a string and a needle.

FIG. 27 shows in detail the cylinder or tube device of FIG. 25.

FIG. 28 shows the lower cylinder prototype of FIGS. 25A and 25B made of steel.

### DESCRIPTION OF THE INVENTION

It is an object of the present invention to provide a magnetic surgical device to manipulate tissue during surgery comprising a grip which allows firmly holding tissue, the grip being preferably made of a nonferromagnetic material; a magnetic system; and means of interconnection between the grip and the magnetic system, this means of interconnection being also preferably made of a nonferromagnetic material. In an embodiment, the grip can consist of a crocodile type clip which comprises two grips linked to each other, the grips having a saw tooth profile on the side facing each other when the grip is closed, and where the grips have fins on the side which is opposite to each other when the clip is closed; a pin linking both grips and allowing to pivot; and a spring keeping these grips in contact in the closed position of the clip.

In another embodiment, the magnetic system can comprise an annular magnet plurality of annular magnets threaded on a lead wire or another pulling element made of a nonferromagnetic material such as plastic (for example a strip) acting as a guide. Preferably the magnets show a very strong magnetic strength and therefore they are preferably made of a rare earth material or any material with high good magnetic features and are preferably gold, nickel or silver plated in order to be inert when contacting the patient's inner organs. In a highly preferable embodiment the magnet or the plurality of magnets may be housed inside a tube-like ferromagnetic steel cylinder. When more than one magnet is housed, these may be aligned in a row or tandem without any attenuation in the magnetic strength of the assembly.

It is another object of the present invention to provide a surgical tool to manipulate a crocodile type clip comprising a main body, comprising a hollow cylindrical tube; a first manipulation end, which comprises a handle with a trigger which is kept separated from the handle by springs or iron straps; a second end for anchoring, which comprises a first grip and a second grip which may pivot respect of the first one; and an insert which runs within the main body and interconnects the first end with the second end so that, when trigger is squeezed, the end grips come together. The tool is preferably made of a nonferromagnetic material to avoid getting magnetized and thereby losing mobility. The second articulated anchoring element works together with the insert which runs inside the main body and with the trigger, so that the latter turns as the trigger is squeezed. The first grip can include a recess at the end to longitudinally receive a grip during manipulation. The first grip can include a lateral recess to receive perpendicularly a grip during manipulation.

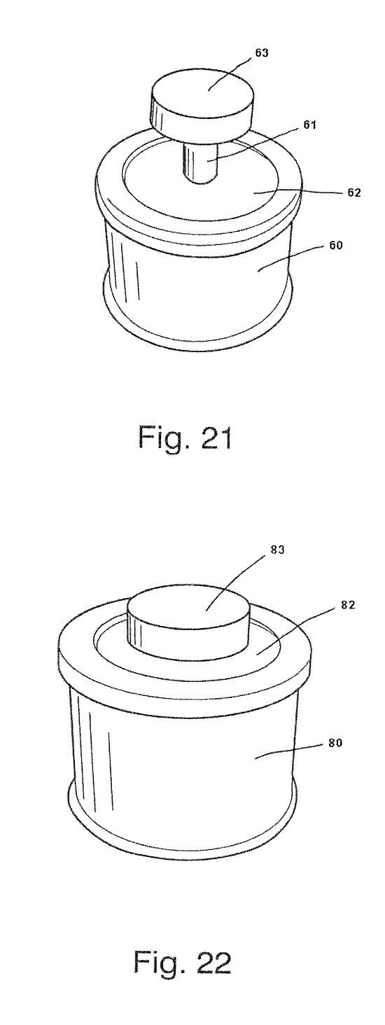

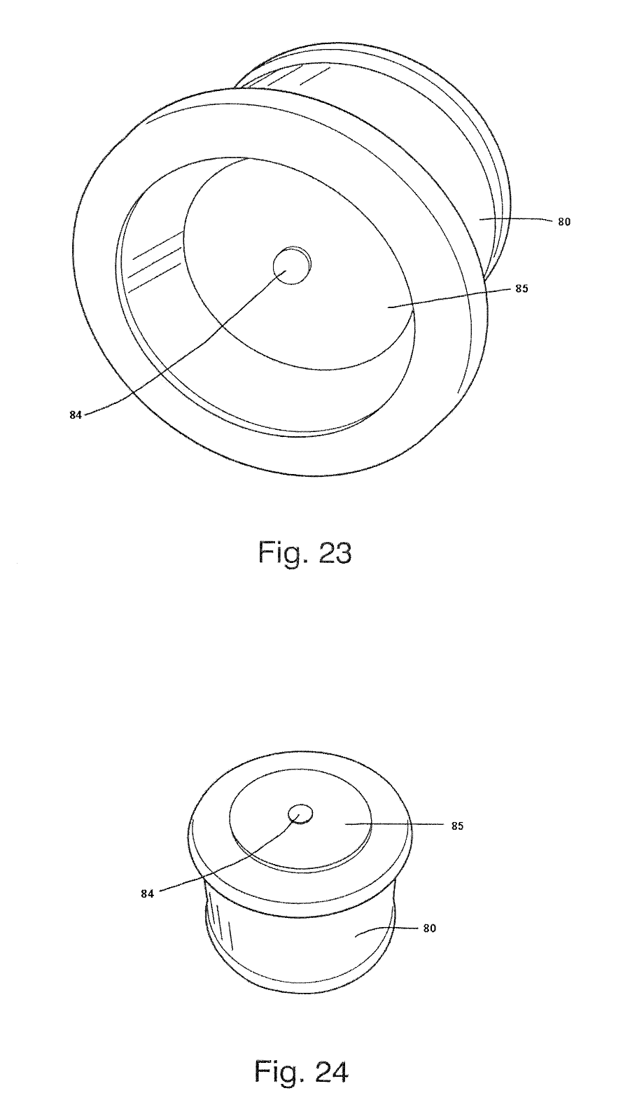

It is another object of the present invention to provide a surgical apparatus for positioning an external magnet during a surgery, comprising a first arm coupled to a fixed point; a second arm, of adjustable length, rotatably connected to the first arm; a third arm, of adjustable length, rotatably connected to the second arm; and a grip rotatably connected to the third arm and which allows trapping an external magnet. In a preferred embodiment, the fixed point where the first arm is linked to, can be at the surgery stretcher. The second arm can comprise holes at both ends so that it is possible to vary the useful length of the second arm when varying the hole in which this one is connected to the first arm and to the third arm. The third arm can comprise holes at an end so that it is possible to vary the usable length of the third arm when varying the hole in which this one is connected to the second arm. The surgical device can be driven in manual or robotic way. Also, in the event the described magnet positioner is not available, the distance of external magnet to the patient's skin can be manually regulated by means of the pot-shaped device or gauntlet mentioned above (see FIGS. 21-23).

It is another object of the present invention to provide a surgical probe comprising at least a magnet on one of its ends. In the embodiment, the surgical probe is hollow and comprises at least a magnet on one of its ends. As already mentioned, the magnets can be made of a rare earth or any other strongly magnetizable material and can be gold, nickel or silver plated. Also probes containing ferromagnetic small steel balls can be used. Placed inside hollow organs, these small balls will be attracted, moved or anchored by magnets which are placed outside the hollow organ, thereby allowing to move or to firmly anchor the organ.

It is another object of the present invention to provide a surgical tool to manipulate spherical magnets or magnets with central holes, comprising a main body comprising a hollow cylindrical tube; a first manipulation end, which comprises a handle with a driving device consisting of two arms articulated one to the other so they can open and close; a second anchoring end, which comprises at least three anchoring elements articulated to each other; and an insert running inside the main body and interconnecting the first end and the second end so that, when the driving device is triggered the two arms articulated to each other open and close, and consequently the anchoring elements at the anchoring come together or are separated. The surgical tool is preferably made of a nonferromagnetic material. The anchoring elements articulated to each other work together with the insert which runs inside of the main body and with the trigger.

It is another object of the present invention to provide a cannula, with a system to fasten preformed knots and for catheter comprising a main body made up by hollow cylindrical tube through which a catheter moves; a first manipulation end, which comprises a handle with an inlet hole to the main body for the entrance of the catheter; a second operative end made up by hollow cylindrical tube, which comprises a lateral hole whereby the thread comes out and a frontal hole at the end whereby the catheter, which had entered by the first manipulation end, comes out, and whereby the thread with preformed external knot comes in. The lateral hole of the second end can be partly beveled and partly comprises an edge so that in use, it allows to fasten a preformed knot. In order to fasten the knot it is necessary to push from the first manipulation end and pull in the opposite sense the thread running outside the cannula, all this through the working canal of the optics.

It is another object of the present invention to provide a surgical organ retractor comprising at least two grips which allow to firmly holding tissue, being these grips preferable made of a nonferromagnetic material; and a interconnection probe between the grips, the interconnection probe being made of a nonferromagnetic material. The grips can consist of crocodile type clips comprising two grips ligated to each other, wherein the grips have a sawtooth profile on the side they are in contact to each other when the clip is closed, and wherein the grips have fins on the side they are not in contact to each other when the clip is closed; a bolt that ligates the grips and allows the articulation between them; and springs or straps which keep the grips in contact in the closed position of the clip.

It is another object of the present invention to provide a method of gallbladder extraction by means of laparoscopic surgery with the use of a single umbilical trocar aided by these devices, comprising the steps of: making a pneumoperitoneum with carbon dioxide at usual pressure; to insert a trocar at umbilical level with closed or opened technique; to insert an optics with working canal through the trocar and to explore the abdominal cavity, then the optics is removed to insert the Dominguez tandem magnetic device through the trocar and then the optics is reinserted. In case of magnets and clip which latter will go through the optics canal, these optics movements could be avoided but probably magnetic field coercive force will be lost and the clip being smaller the ability to hold thicker tissues will be lost. After inserting a Dominguez tandem into the abdominal cavity through the trocar and by means Thomas pliers which enters by the optics operating canal, to see and to follow the positioning of the tandem that will be attracted by the magnetic field generated by an external magnet; the bottom of gallbladder is gripped with the tandem clip, aided in doing so by Thomas pliers, thus pulling the vesicular bottom towards cephalic over the liver and towards the patient right shoulder, this being achieved changing the position of the external magnet. To insert another Dominguez tandem through the trocar by means of a Thomas pliers; to position the Dominguez tandem using an external magnet leaving the crocodile type clip with its end directed towards the gallbladder; to take the gallbladder in infundibulum or Hartmann sac by opening of the crocodile type clip with Thomas pliers; to position the gallbladder properly by moving another external magnet which attracts by magnetic field the Dominguez tandem inside the patient; to move the Dominguez tandem towards the flank and towards the patient right iliac fossa to expose the Calot triangle that contains the cystic artery and the cystic conduit surrounded by peritoneum; to dissect the cystic conduit and the cystic artery with instruments which enter by the working canal; to repair the cystic conduit with external tie by means of a knot without fastening, one the ends of the thread coming out by the trocar; to make cysticotomy of the cystic conduit with scissors; to catheterize the cystic conduit by the working canal by using a Valentina cannula fastening the catheter through the hole of Valentina cannula avoiding in this way the contrast fluid reflux and catheter displacement; the study is made, then the catheter is removed and the fastening of the knot is finished with Valentina cannula; optionally use clip. Clip or ligate the artery and section it; separate the gallbladder from the hepatic bed by dissection with instruments inserted by the optics canal for example the electro-scalpel and by moving the external magnets for the presentation of the gallbladder until the definitive separation of the gallbladder from the liver is achieved. Release the crocodile type clips with the Thomas pliers; take the cystic remaining that is left by the side of the gallbladder using the Thomas pliers; remove the Dominguez tandem once it is released from the magnetic field of the external magnet; to wash, to aspirate and to control for haemostasis; and to vent CO₂ by umbilical trocar and close. In addition, in the step of positioning the gallbladder by moving an external magnet, this can be made also by descending, instead of a Dominguez tandem, a magnet with a central hole by means of a point in U with external-internal-external thread which is passed during its internal route, through the gallbladder and its two ends remain external to the umbilical trocar, then a magnet with hole is threaded to one of the ends and a sliding knot is made (Gea or Roeder knot) behind the magnet in such a way that with a clamp to fasten knots, we slide the magnet towards the vesicular bottom.

In case of gallbladder under pressure, before any maneuver it is previously vented by puncture and aspiration by the working canal. In case of thickened vesicular wall or escleraotrofic gallbladder, if the crocodile type clip cannot take the vesicular wall a magnet is inserted by means of U point transparietal to the gallbladder. Once the gallbladder is pulled with an external magnet, if adhesions exist they are treated with scissors or electro-scalpel. In the step of moving the Dominguez tandem towards the flank to expose the Calot triangle, if necessary, more Dominguez tandem are placed for greater tractive force and exhibition of the triangle. If the liver does not allow an easy dissection of the peritoneum and of the elements of the triangle, a William retractor is used or a needle with blunt end “string carrier” can be used, placed at right hypocondrium. In the step of catheterizing the cystic conduit, this can be made by introducing the catheter in percutaneous way, holding the same within the cystic with prehensile clamp by the canal. If string carrier needle is used it is possible to catheterize by this via and it is fastened within the cystic by prehensile clamp through the canal. In the step of removing the catheter and fastening the knot with Valentina cannula it is possible to place Hem-o-lok type clips to assure the closing of the conduit before its definitive section. If calculus in the biliary tract are found, use Dormina type baskets or are progressed to the duodenum by placing a spherical magnet in choledochus, and then by means of a capillary end containing a magnet, the espheric magnet is slid, dragging the calculum. In the step of removing the gallbladder taken by the remaining cystic, a sterile bag can be inserted and positioned with the crocodile type clip that was placed in infundibulum and a Thomas pliers by the canal, introducing then the gallbladder in bag and removing it so avoiding umbilical contamination.

It is another object of the present invention to provide a method of performing surgery of Hiatus comprising making a pneumoperitoneum with carbon dioxide at usual pressure; to insert to trocar at umbilical level; to insert an optics with working canal through the to trocar and to explore the abdominal cavity, to remove the optics to insert a Dominguez tandem by means of a Thomas pliers through the trocar and the optics so that the crocodile type clip will grip the stomach, the crocodile type clip being surrounded with a cover that avoids damage to the stomach; to position the Dominguez tandem by means of the usage of an external magnet remaining the crocodile type clip with its end directed towards the stomach; to insert a William retractor through the umbilical trocar by a Thomas pliers; to separate the left hepatic lobe by means of the William retractor to so expose the gastric esophagus union and the diaphragmatic pillars; to insert a surgical probe comprising at least one magnet at one of its ends through the mouth, positioning it in the stomach; to dissect and to close the pillars of the diaphragm by dissecting the esophagus and the esophagus-gastric union; to make a funduplicature to avoid the elevation of the stomach to the thorax and to avoid the hydrochlorate acid reflux from the stomach towards the esophagus by incompetence of the lower esophagic sphincter; to adjust the pillars and funduplicature by means of extracorporeal needles with thread and knots and/or continuous o separated sutures; to remove the surgical probe of the stomach; to remove Williams retractor; to remove the Dominguez tandem once it is released from the magnetic field of the external magnet; to wash, to aspirate and to control haemostasis; to vent CO₂ by the umbilical trocar and to close. In addition, if a hernia is detected, it is reduced. If diverticulm is detected, a magnet is placed inside the same by endoscopy and with an external magnet we mobilize it for its dissection outside of the esophagic wall in order to latter perform a resection on it and a to suture the esophagus. Optionally, in the fundiplicature the short vessels running from the stomach to the spleen are sectioned to allow a greater mobility of the stomach in the maneuvers before the funduplicature, and a suture stomach-esophagus-stomach is performed thus creating a valve that is calibrated with a surgical probe with magnets inserted through the mouth. It is further possible to insert additional trocars by usual sites to use usual laparoscopy instruments.

It is another object of the present invention to provide a method for performing spleen surgery or splenectomy comprising: to perform a pneumoperitoneum with carbon dioxide at usual pressure; to insert trocar at umbilical level; to insert an optics with working canal through the to trocar; to insert a Dominguez tandem by means of a Thomas pliers through the trocar so that the crocodile type clip will grip the stomach, the crocodile type clip being surrounded at its toothed end with a cover that avoids damage to the stomach; to position the Dominguez tandem by means of the usage of an external magnet. To place another Dominguez tandem the crocodile type clip remaining with its end directed towards the spleen ligament to take the ligament aided by the Thomas pliers positioning with another external magnet until its traction is achieved; to insert a surgical probe comprising at least a magnet at one of its ends through the mouth positioning it in the stomach; to section all the ligaments connecting and anchoring the spleen, wherein the section is made on the ligament between spleen and tandem by cutting clamp entering by umbilical trocar; to release pedicle or splenius hilus whereby artery or arteries and vessel or vessels enter the spleen; to remove completely the released spleen by the navel placing it in a protective bag; to remove the surgical probe from the stomach; to remove the Dominguez tandem once it is released from the magnetic field of the external magnet; to wash, to aspirate and to control haemostasis; to vent CO₂ by the umbilical trocar and to close. It is possible to use a Williams' separator to separate left hepatic lobe in case this lobe does not allow the vision of spleen or of the spleen ligaments. It is possible to use another Williams' separator to hold the spleen and so achieve a better counter-traction thereof when the ligaments are released. It is possible to use one or more Dominguez tandem surrounded by a cover to manipulate, with the aid of an external magnet, the colon, intestines or stomach for better exhibition of the surgical field. In addition, it is possible to triturate the completely released spleen within a bag to remove it by the navel.

It is another object of the present invention to provide the usage of one or more magnetic surgical devices to manipulate tissue in appendix and colon surgeries, a surgical tool to manipulate a crocodile type clip, one or more external magnets for handling mesoappendix-mesocolon, a surgical apparatus to position an external magnet during surgery, and a surgical probe comprising at least one magnet at one of its ends which is inserted through anus in the colon.

It is another object of the present invention to provide the usage of one or more magnetic surgical devices for gynecological surgeries placed by umbilical trocar and anchored to the uterus to manipulate the ovaries, the tubes, the uterus or infundibulum, a surgical tool to manipulate a crocodile type clip, one or more external magnets to drive the surgical magnetic devices, a surgical apparatus to position an external magnet during surgery, and an organ surgical retractor.

It is another object of the present invention to provide the usage, in abdominal wall surgeries, of one or more magnetic surgical devices for handling the peritoneum, a surgical tool to manipulate a type crocodile clip, one or more magnets to manipulate the surgical magnetic devices, and a surgical apparatus to position an external magnet during surgery.

It is another object of the present invention to provide the usage, in urologic surgeries, of one or more magnetic surgical devices for handling of the kidney, a surgical tool to manipulate a crocodile type clip, one or more external magnets to manipulate the surgical magnetic devices, a surgical apparatus to position an external magnet during surgery, a surgical probe comprising at least one magnet in one of its ends for placing in ureter by cystoscopy for location, identification and handling of the same, and a organ surgical retractor.

It is another object of the present invention to provide the usage, in achalasia surgeries, of one or more magnetic surgical devices, a surgical tool to manipulate a crocodile type clip, one or more external magnets to manipulate the surgical magnetic devices, a surgical apparatus to position an external magnet during surgery, a surgical probe comprising at least one magnet in one of its ends, and an organ surgical retractor.

It is another object of the present invention to provide the usage, in diverticles surgeries, of one or more magnetic surgical devices, a surgical tool to manipulate a crocodile type clip, one or more external magnets to manipulate the surgical magnetic devices, a surgical apparatus to position an external magnet during surgery, a surgical probe comprising at least one magnet in one of its ends, and an organ surgical retractor.

It is another object of the present invention to provide an auxiliary assembly for surgery carried out with the usage of magnets, comprising a clip, a string and a needle.

### DETAILED DESCRIPTION OF THE INVENTION

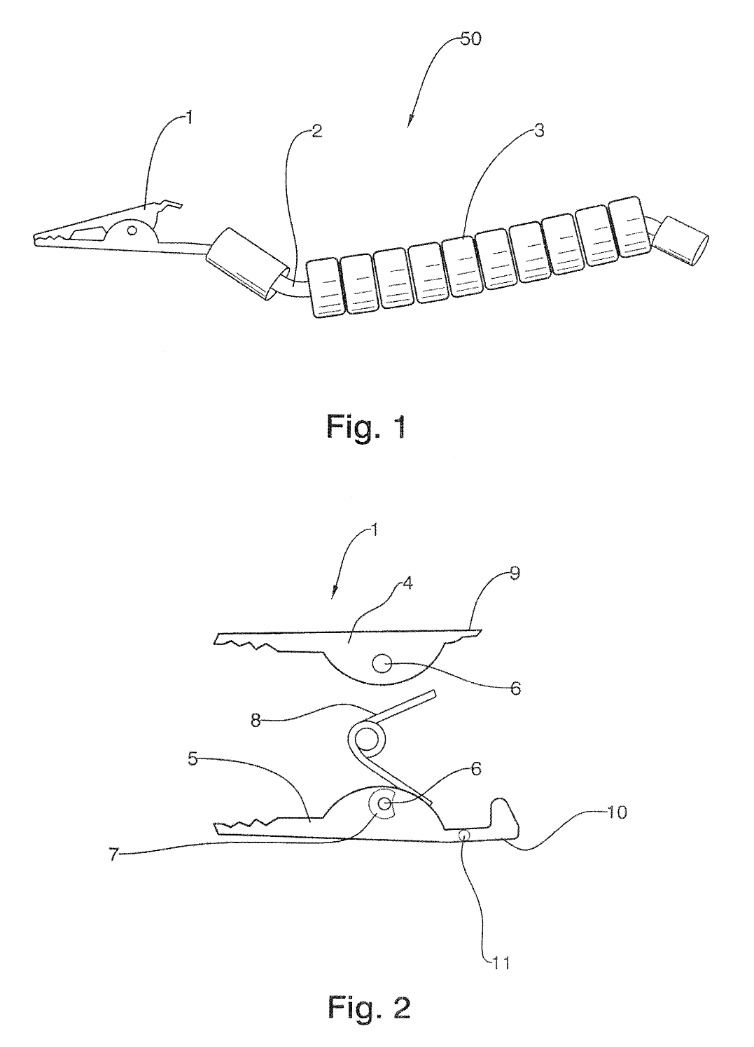

FIG. 1 shows a first embodiment of the present invention, which comprises a magnetic surgical device for manipulating tissue 50 during a surgery (herein called Dominguez tandem) made up by a crocodile type clip 1, which is coupled by means of a wire 2 or alternatively another equivalent traction element (not shown in the figure) such as a strap, with a magnet array 3. Clip 1 and all its components and the wire 2 or strap are preferably made of non magnetizable surgical steel or a nonferromagnetic biocompatible material, preferably a biocompatible plastic material. In an alternative embodiment the wire and or the strap may be also made of a magnetizable material.

Preferably, clips 1 should be smaller than 10 mm, of 10 to 50 mm in length. Preferably, the non-magnetic material can be Steel SAE 304 or 316 L or any other biocompatible material such as acetate of polyvinyl, titanium, etc.

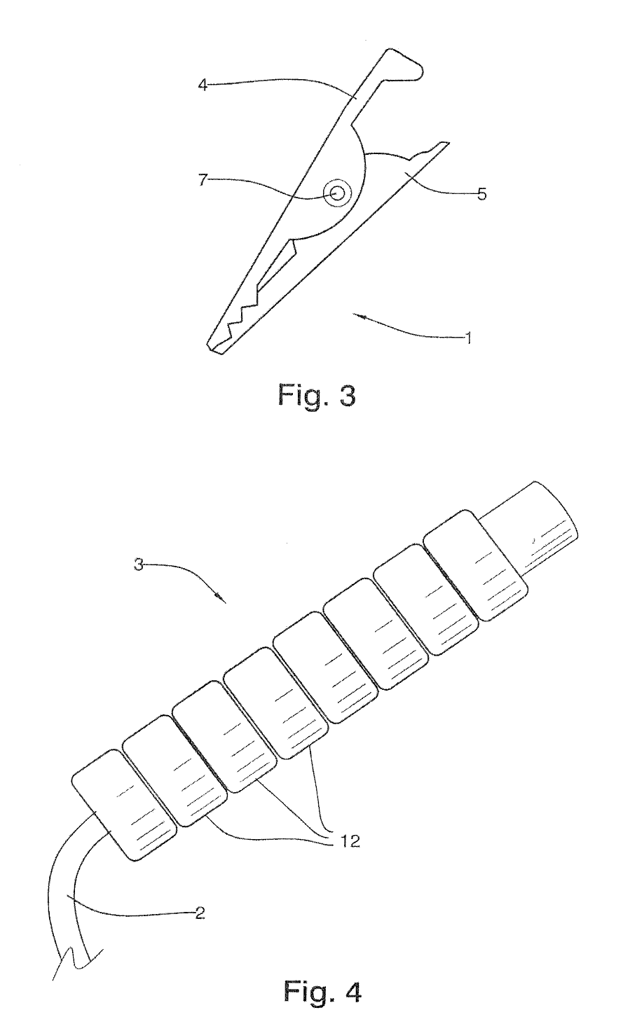

FIG. 2 shows an exploded view of the crocodile type clip 1. The clip comprises two grips 4, 5, which have in one of their ends, a sawtooth profile to facilitate gripping the human organ by the clamp. Both grips 4, 5 have a through hole 6 by which they are linked with an anchoring bolt 7, which allows rotation movement between both grips 4, 5. Clip 1 comprises in addition a spring 8 that biases the grips 4, 5 to a closed position when the user exerts no force is on them.

In addition, grips 4, 5 comprise fins 9, 10 at opposite ends. These fins are designed to allow a force exerted on them to overcome the force of spring 8 and to cause the grips 4, 5 to rotate around the anchoring bolt 7 to open. Thus, the open clip is positioned on the human organ to be anchored, in order to further release fins 9, 10 and allowing the spring 8 to close again grips 4, 5 on the organ. The grip 10 in addition comprises a through-hole 11 in which a wire, a non ferromagnetic thread or a string is inserted. As explained above the wire, thread or string may be also replaced by a strap, preferably a plastic strap.

FIG. 3 shows to the crocodile type clip of FIG. 2 in closed position. Here it may be seen how the grips 4, 5 remain closed by the action of spring 8 (not shown).

FIG. 4 shows the magnet array 3 made up by a plurality of annular magnets 12. The magnets are built with any rare earth material which showing excellent magnetic features, such as high magnetic moment, and preferably with neodymium, these are and gold, nickel or silver plated. The magnets can have different shape and size, with or without a hole. The plurality of magnets is kept together by magnetic attraction if they do not include a central hole. Generally, it is preferable for them to be smaller of circular profile and smaller than 10 mm to be introduced into the abdominal cavity by a trocar or by natural holes. It is possible to insert them alone, guided by threads, strings, straps or probes or by means or pliers. The annular magnets should be threaded on a wire, a string or a strap made of a non magnetic material.

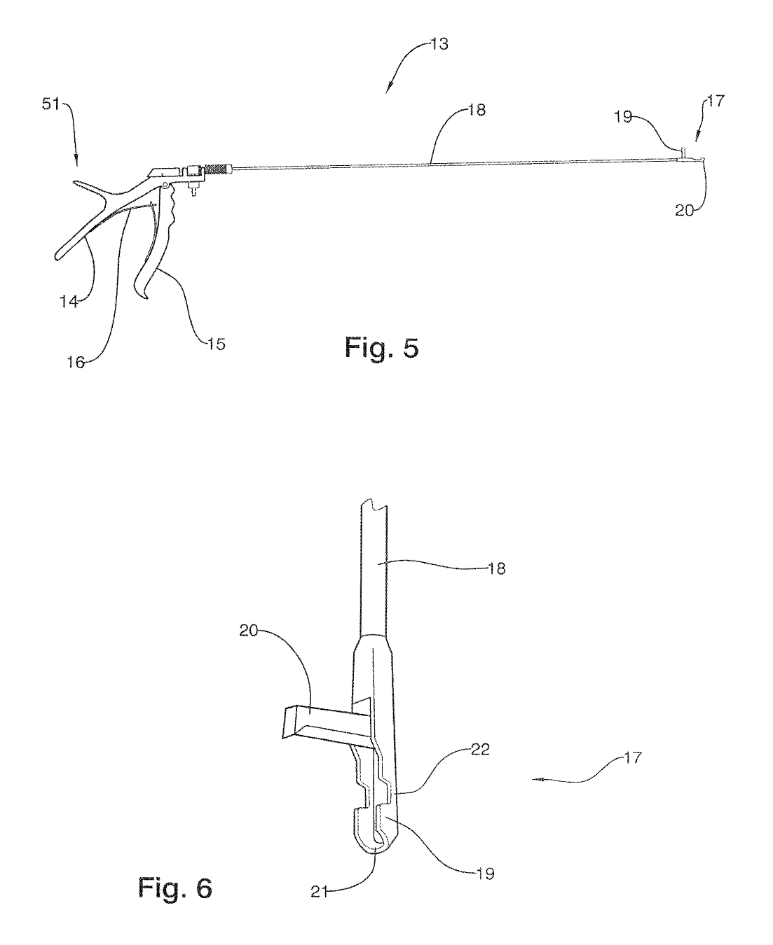

FIG. 5 shows pliers 13 (herein called Thomas pliers) designed to manipulate clips 1. The Thomas pliers 13 consists of a hollow cylindrical tube 18 through which runs an insert (not shown) that links both ends of the pliers. In one of its ends 51, the Thomas pliers 13 comprises a trigger 14 with a handle 15 which is kept apart form trigger 14 by means of springs or straps 16. At the opposite end 17, the Thomas pliers 13 comprises a pair of anchoring elements 19, 20 so that when the operator exerts pressure on handle 15 at the end 51 of the Thomas pliers, the anchoring elements 19, 20 at the other end 17 of the clamp are joined due to the displacement of the inner insert to remain en position of anchoring clip 1.

FIG. 6 shows in detail the end 17 of the Thomas pliers 13. It may be seen that the anchoring element 19 is fixed to, and moves with, the hollow main body 18. Also, the anchoring element 20 is articulated so that it may be closed and opened with respect to the anchoring element 19, when the insert that runs inside hollow cylindrical tube 18 is actuated by the trigger 14. The profile of the anchoring elements 19, 20 is adapted to be able to manipulate clips 1. For achieving so, the anchoring element 19 comprises a recess 21 which allows grabbing clip 1 from behind. In order to being able to grab clip 1 perpendicularly, the anchoring end 19 comprises a side recess 22. The Thomas pliers 13 is able to anchor, to open, to grip from the side, from the front grip and from behind the clips 13.

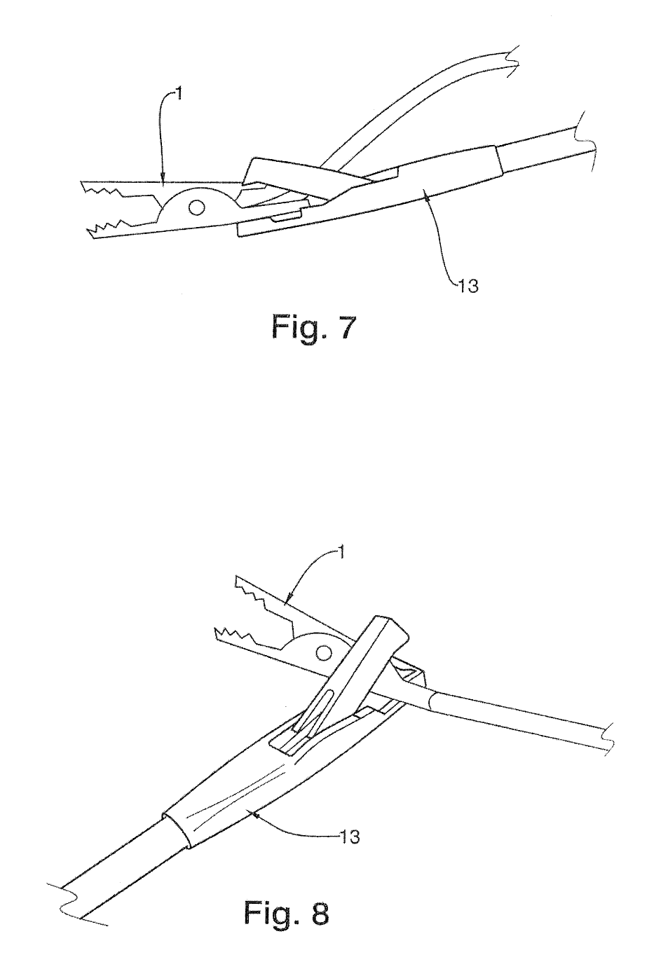

FIG. 7 shows how in practice Thomas pliers 13 holds clip 1 from the back. FIG. 8 shows in practice the perpendicular anchoring of clip 1. In use, the end of the Thomas pliers comprising trigger 14 and handle 15 remains outside the abdominal cavity, whereas the end 17 comprising anchoring elements 19, 20 is inserted in the abdominal cavity.

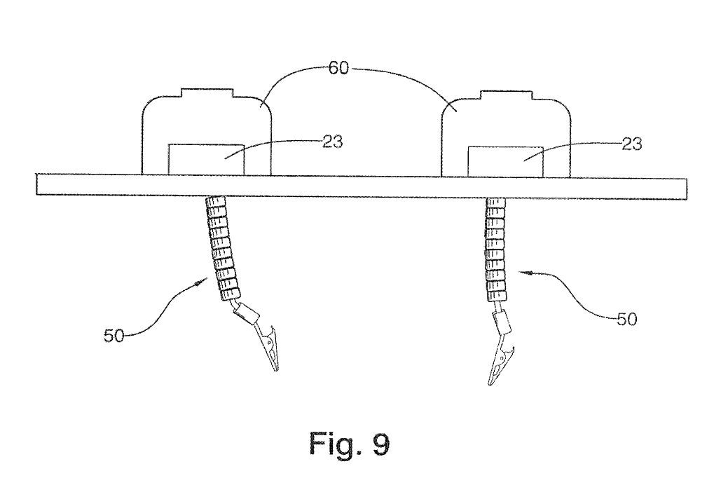

FIG. 9 shows a pair of pot-shaped supports or gauntlets 60, each of which houses an external magnet 23 that is used to manipulate a Dominguez tandem 50, which in use during surgery is within the human body. External magnets 23 can be permanent magnets or electromagnets; they should generate a magnetic field enough for the action required by the surgeon. The dimensions of magnet 23 will depend on the required use. In a preferred but not limiting embodiment, its external magnet can be 50×50×25 mm in size.

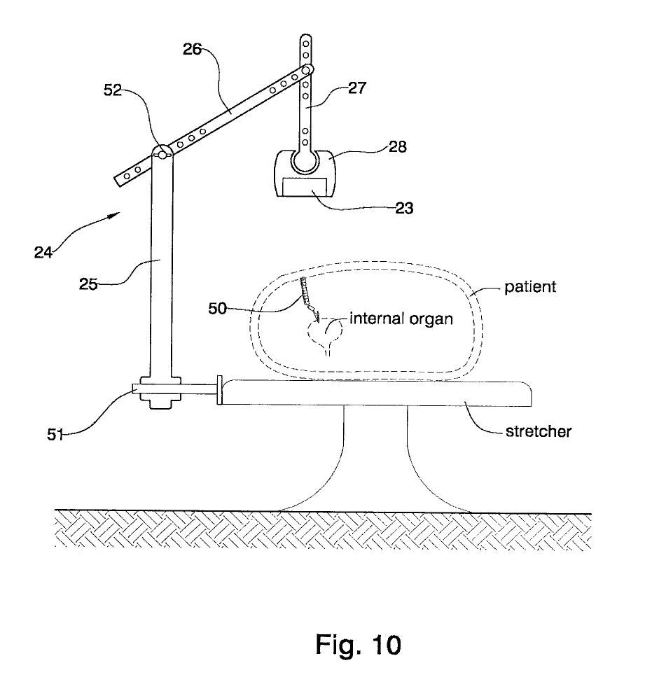

FIG. 10 shows a mechanical apparatus 24 with a rotation up to 360° wide, which is useful for moving the external magnet 23 during surgery. In a preferred embodiment, the mechanical arm comprises a first arm 25 which can be coupled to the surgery stretcher or any other point designed to this purpose, a second arm with holes 26 which allows to regulate the reach range of the articulated set, and a third arm with holes 27 that holds the external magnet 23 through a grip 28. First arm 25 is coupled to the patient's stretcher, or any other fixed point, via a fastening device 51. At the other end, first arm 25 is coupled to the second arm 26 through a regulating device 52. This mechanical arm can be manual or robotic.

FIG. 11 shows a probe 29 comprising a plurality of magnets at one of its ends. This probe is used together with the Dominguez tandem 50 to manipulate hollow organs like stomach, intestine, etc. In a first embodiment, probe 29 is hollow and comprises a plurality of magnets 30 inside one of its ends.

In laparoscopic surgery, the probes are inserted in the human body through some natural hole, preferably the mouth, the anus or the vagina. On the other hand, through the optics canal, Thomas pliers 13 is inserted holding the set made up by a crocodile type clip 1 and a magnet array 3. Further, probe 29 is arranged in magnetic contact by means of the magnetic attraction between magnets 30 of the probe with magnets 3 of system 50, so that the human tissue wall will be sandwiched between both magnet systems, thereby being able to be properly manipulated and anchoring the free clip 1 of system 50 in the site selected by surgeon by anchoring all the set, aided by the Thomas pliers 13. Magnets 30, can be gold or nickel plated, can show different shapes, diameters and sizes, so as to be easily swallowed with water or to be inserted by natural holes with or without the use of endoscopies.

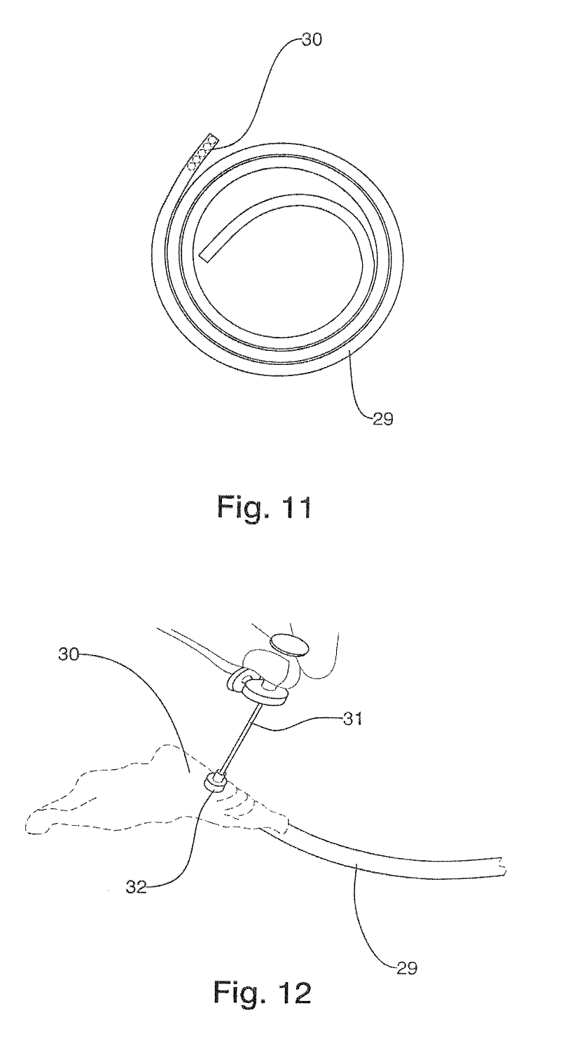

FIG. 12 shows the use in practice, of the probe in FIG. 11. In the example, the probe is used together with a needle 31 comprising at its end a bell-shaped element with a magnet 32. Needle 31 is inserted into the human body through the abdominal wall. Then, probe 29 and the magnet array 30 are arranged in magnetic contact with the needle 31 which holds at its end a magnet 32, so that the human tissue wall remain sandwiched between both magnet arrays 30, 32, thereby being able to move it properly. Needle 31 can be made of a steel SAE 304 or 316L and 1 mm in diameter. The bell-shaped element containing magnet 32 allows introducing needle 31 and may be made of steel 304 IS or 316L.

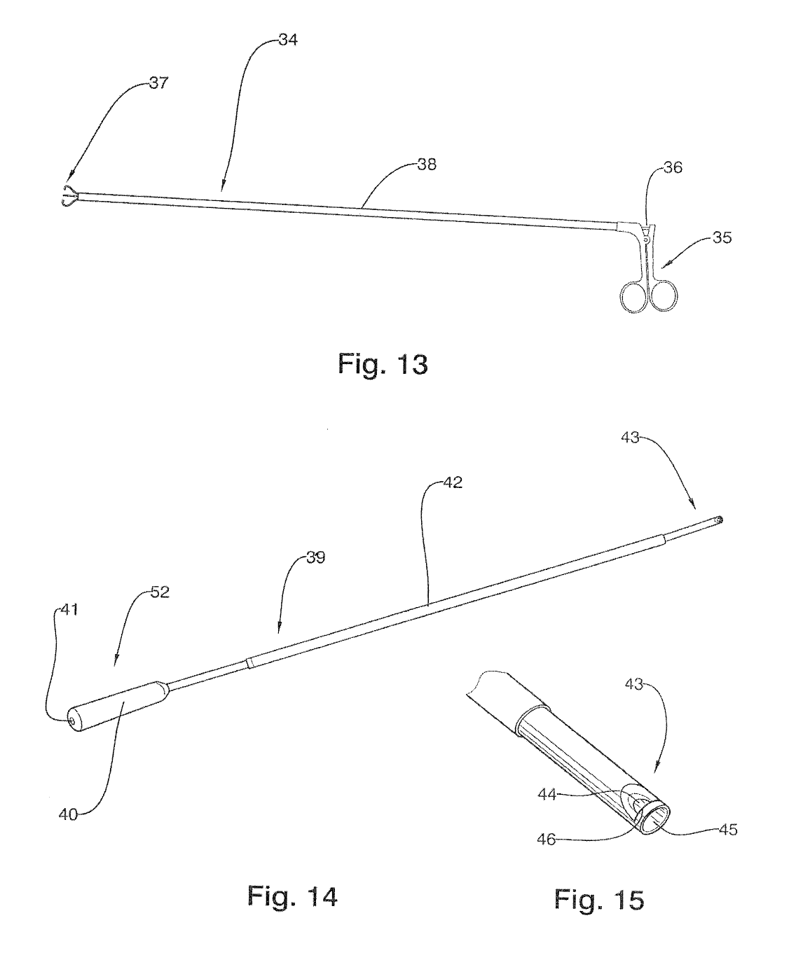

FIG. 13 shows pliers 34 (herein called Camila pliers) within the optical surgery canal and the set within the trocar, which is used to manipulate spherical magnets or magnets having central holes. Camila pliers 34 comprises a handle 35, a hollow cylinder 38 through which an insert 36 moves and engaging handle 35 by the side and being cut at the other end thus bearing three ends 37 that can be opened or closed when handle 35 is driven. The material used is preferably a non-ferromagnetic surgical steel. In a preferred embodiment, but not limited to this version, the dimensions of Camila pliers 34 may be 5 mm in diameter by 45 cm in length. Handle 35 can close or open the 3 ends 37, thereby holding spherical magnets or washer-shaped magnets with central holes. These pliers are useful for placing or removing spherical magnets and washers, or even the set of crocodile clamp 1 with magnets 3, into or out of the abdominal cavity.

FIG. 14 shows a cannula 39 (herein called Valentina cannula) that is used for carrying out cholangiography (study of the biliary tract). The Valentina cannula 39 comprises a first end 52, a second operative end 43 and a hollow main body 42. The first end 52 comprises a handle 40 with an inlet hole 41 for the entrance of a catheter. FIG. 15 shows the second operative end 43 of a Valentina cannula 39 in detail, which is inserted into a patient. The end 43 comprises a side beveled hole 44 and a front hole 45. Side hole 44 comprises an edge 46 that fastens a pre-formed knot. The catheter comes out and the thread enters into the hole 45 and the thread comes out through lateral hole 44. The upper edge 46 is useful for making sure that the knot is fastened when the catheter is within the cystic conduit and the pre-formed knot is outside the end, as the thread is pulled from outside and the Valentina cannula is pushed.

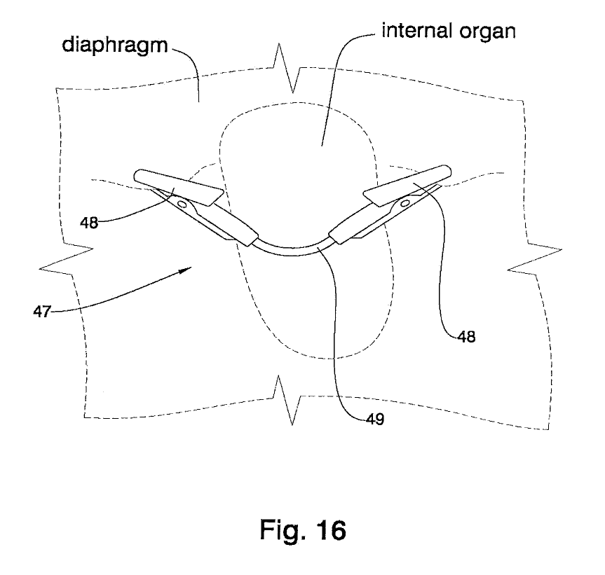

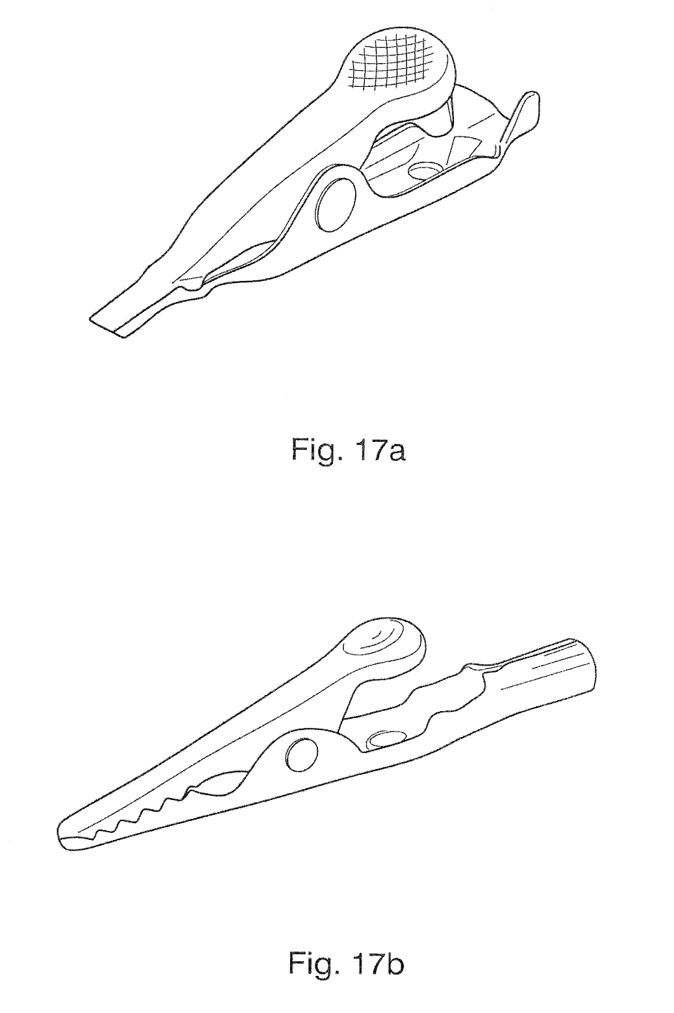

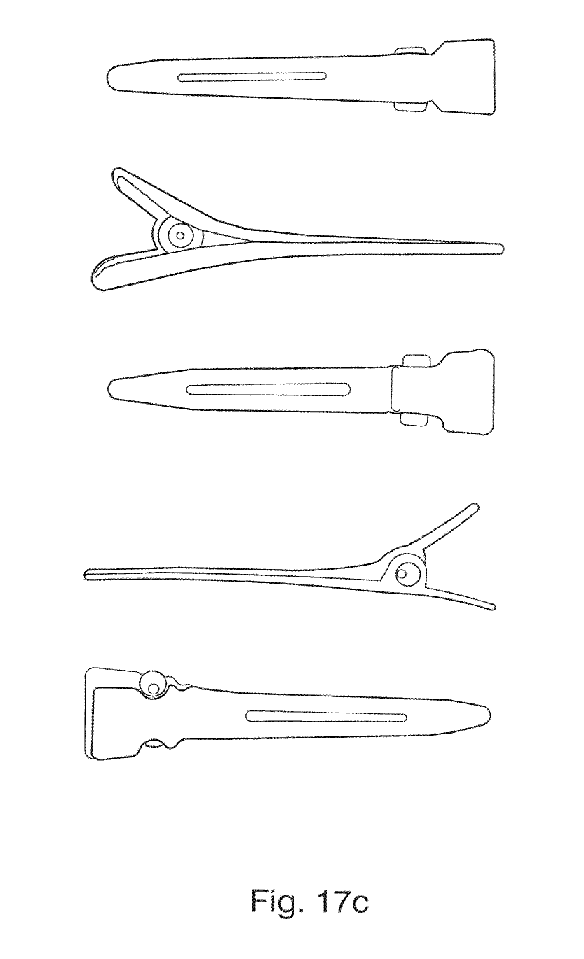

FIG. 16 shows a retractor or separator 47 (herein called William retractor or separator), which is used to retract organs during surgery for having thereby a better access to the organ that is being operated. Thus, in certain surgeries, to be able to see what is being operated, it is necessary to separate, for example, the liver, the uterus, the spleen, etc. William retractor 47 consists of two clips 48 linked by a probe 49 of convenient length, which works as a retractor for the liver, spleen, uterus, etc. The organ lays on probe 49 and the clips 48 are set at several points, for example diaphragm-peritoneum for biasing and supporting the left hepatic lobe. This maneuver allows to see the hiatus in case of hiatus hernia surgery, achalasia.

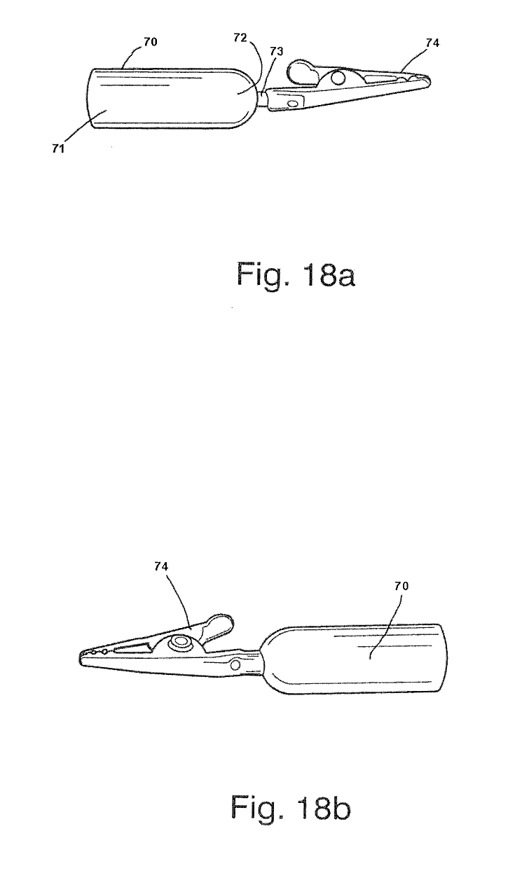

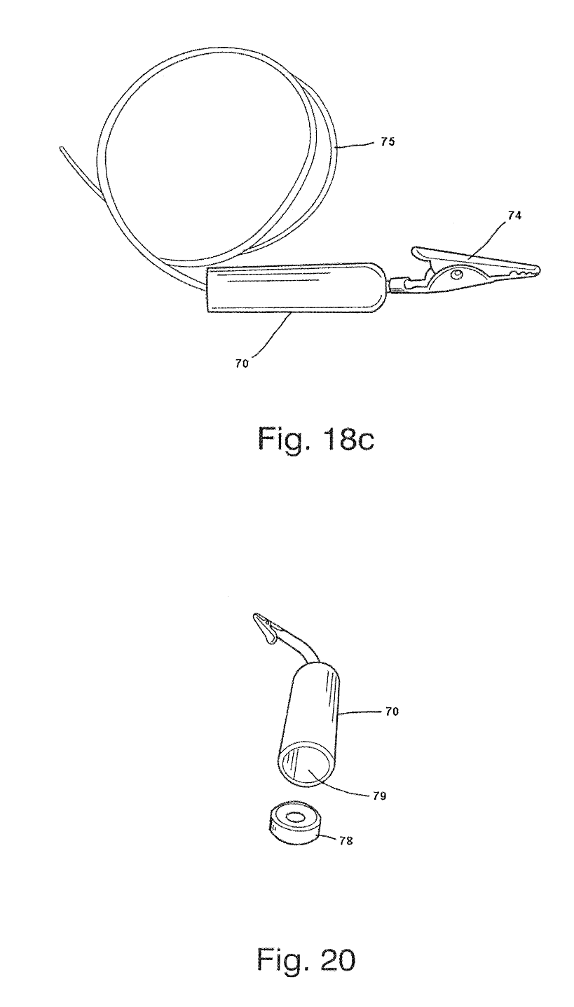

FIGS. 18A, 18B and 18C show a preferred embodiment in which one magnet or a plurality of magnets (not shown) are covered by a magnetic steel tube 70. The tube 70 has a first cavity 71 for housing the magnet or tandem of magnets and another second cavity 72 for housing a guide or interconnection means 73 that link the tube 70 with a fastening means 74. The second cavity 72 allows anchoring a string or a thread 75 as a safety feature for avoiding the tube from accidentally falling during a surgery intervention, and also for facilitating its placing and removal from the patient. Preferably, the tube 70 is made of ferromagnetic (magnetizable) steel 430 so that the magnet or the tandem of magnets achieve higher magnetic strength.

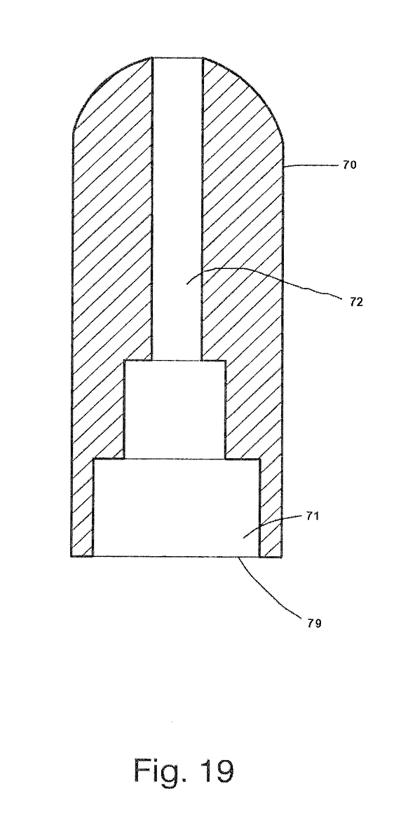

FIG. 19 shows that the annular magnet (not shown) is housed in the first cavity 71 with 8 mm internal diameter and through which the string or thread (not shown) is passed; in the second cavity 72 with 5 mm internal diameter a plastic header (not shown) is placed linking the magnet tandem to the clip and which can be firmly tied to the string coming out of the tube. If the magnet has no central hole, the string can come out through a side hole next to the header. In the event that two magnetized devices come together inside the patient's body, thanks to the help of the string and the tube it should be much easier to separate these.

FIG. 20 shows more clearly the opening 79 through which the one or more magnets 78 are inserted inside the tube.

FIGS. 21-24 show an apparatus that helps keeping an exterior magnet at a stable and regulated height over the patient's skin. It consists of a cup-shaped device 80 with a tap 82 that has a central threaded through-hole to which a pin 81 is pivotally fixed. The pin 81 comprises a manually driven solid wheel 82 in the upper end and a magnet assembly 85 rigidly fixed via a screw 84 to the lower end. The pin 82 and the magnet assembly 85 are lifted or lowered by turning the wheel 82 clockwise or counterclockwise, thus regulating the magnet's height when the pin screws into or out of the cup-shaped element. In an alternative embodiment, this regulation can also be done electrically or automatically by linking the regulating device to an arm fixed to the patient's stretcher.

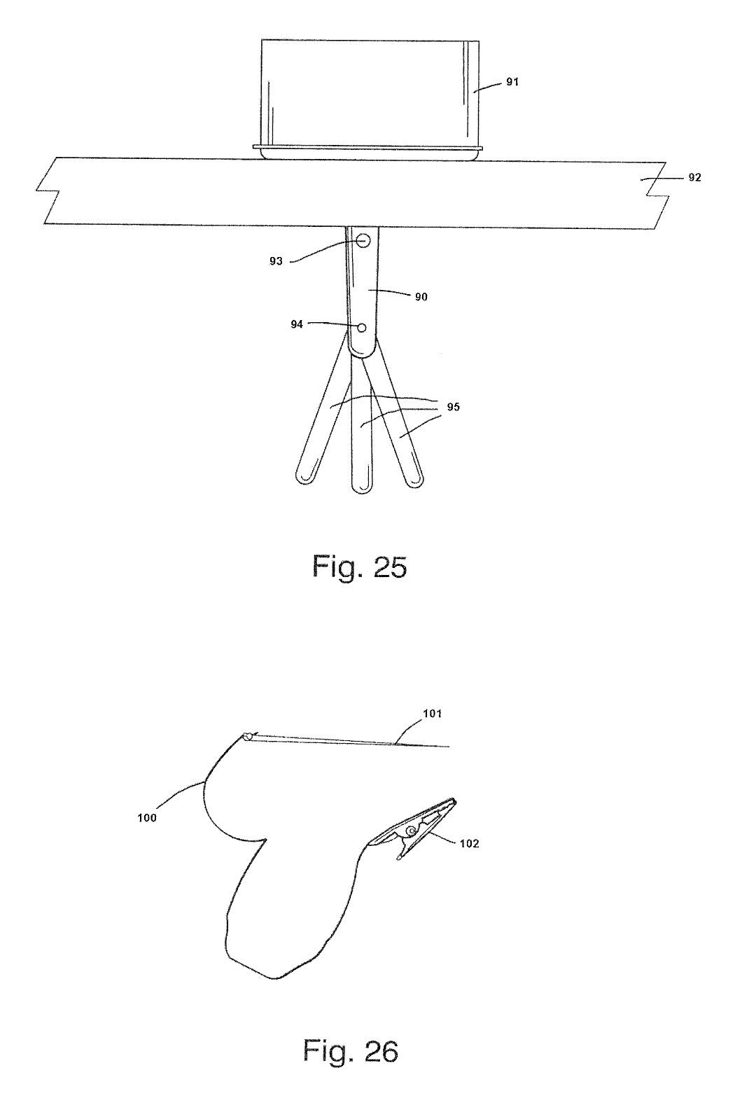

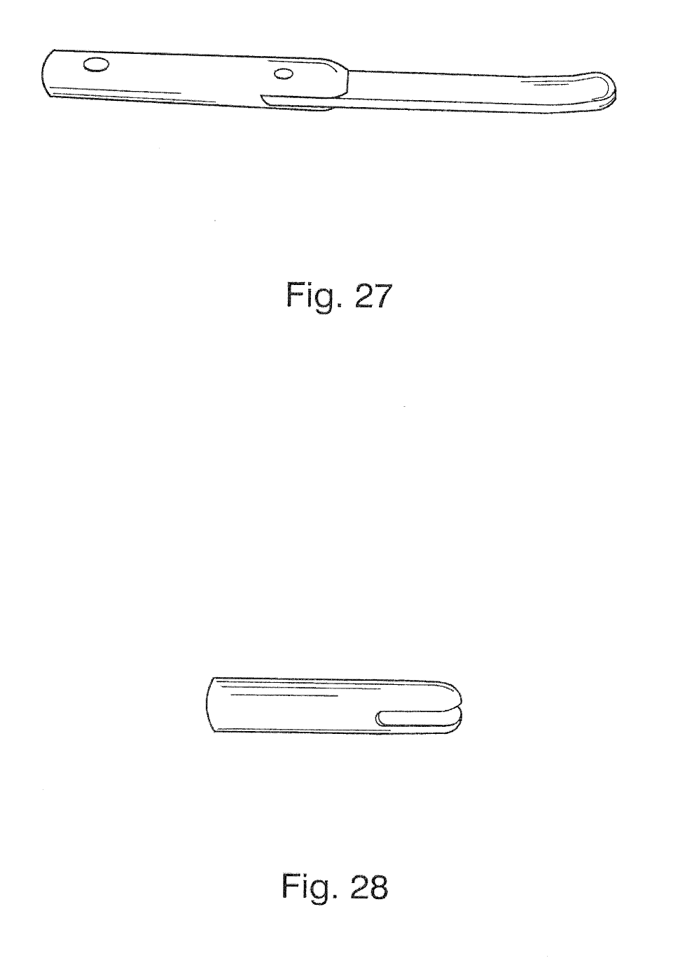

FIGS. 25, 27 and 28 show another embodiment of the hepatic separator. In FIG. 25 it may be seen an upper rigid plastic base 92, in this figure in white colour, over which lies the external magnet (not shown for being inside a protection flexible paper or plastic white housing 91) but the latter element is not part of the device and is only shown for housing the magnet. Below the plastic base it may be seen a device formed by a cylinder 90 projecting downwards which is the element that will be inserted into the patient. The external magnet is placed on the base 91 and attracts the cylinder 90 which is below the base and, in between, simulating the presence of human skin was placed a flexible sheet of plastic 92. The cylinder 90 may be made of magnetizable steel or else of a biocompatible plastic material (not magnetizable) and includes two holes; an upper one 93 for inserting a retention pin and a lower one 94 for receiving a lower pin and both holes being internally connected by a longitudinal channel which houses a spring not shown in the figure). The lower pin acts as a rotation axis of three metal legs 95 forming an angle and these tend to open biased by the spring. The surgeon closes the metal legs against the spring's force as shown in FIG. 27 before inserting the device in the patient's body. Once inside, the metal legs open again and act as separators. FIGS. 27 and 28 show further details of the way the device is manufactured. FIG. 25 also shows that the external magnet 91 can easily pull and keep in place the lower steel cylinder 90 by magnetic interaction.

FIG. 26 shows another device comprising a string 100 linking a needle 101 and a clip 102. In use, the clip 102 grabs the inner organ while the needle 101 and the string 100 pass come out through the patient's abdominal wall. Once outside, the surgeon controls the organ's position by means of the string 100. In a preferred embodiment, the clip 102 is made of a magentizable material, by which the clip 102 might acts as a magnet or a magnet tandem and fulfils functions similar to those already described before by these elements. In another preferred embodiment, the clip 102 is made of non magentizable material, and its sole function is to grab the inner organs in a mechanical way.

Several surgical techniques developed from previously described instruments are described bellow.

Cholecystectomy or Gallbladder Removal by Means of Laparoscopic Surgery with the Use of a Single Umbilical Trocar.

1—Introduction of trocar at umbilical level by means of closed or opened technique according to the surgeon's preference. Pneumoperitoneum with Veress' needle with carbon dioxide at usual pressure for closed technique. Optics with working canal is placed. Introduction of Dominguez tandem 50 (FIG. 1) which comprises magnets 3 in abdominal cavity by trocar. The Dominguez tandem can or cannot be taken by Thomas pliers 13 (FIG. 5) and tracked under direct vision by the optics.

2—Traction of the gallbladder's bottom towards cephalic and over the liver, and towards the patient's right shoulder: Dominguez tandem 50 is inserted by trocar. Once placed in the cavity, the Dominguez tandem 50 is positioned by means of use of an external magnet 23 (FIG. 9) and keeping the crocodile clip 1 with its end directed towards the gallbladder. With Thomas pliers 13 the opening of the clip 1 takes place and the gallbladder bottom is grabbed. By moving the external magnet 23 which attracts by magnetic field the magnet array 3 inside the patient, the gallbladder is positioned as desired. This can also be done by lowering a magnet having a central hole by means of a thread that passes first through the organ and both ends remain outside, a magnet having a hole similar to a washer is threaded, and a Gea-Roeder knot or any sliding knot running in a single sense and fastening the thread with the corresponding fastening-knots tool to the vesicular bottom.

In case of pressurized gallbladder, before doing any maneuver it can be previously vented by puncturing and aspirating via the working canal, in case of verifying the existence of a thickened vesicular wall or escleraotrofic gallbladder. If the crocodile type clip 1 cannot take the vesicular wall, a magnet is lowered as described above, by means of transparietal point to the gallbladder. Once the gallbladder is pulled by an external magnet 23, if adhered tissues exist they are separated in this step with scissors or electro-scalpel.

3—Exposure of Calot triangle: Another Dominguez tandem 50 similar to the previously mentioned one will take infundibulus or Hartmann sac and is moved with another external magnet 23, the assistant moves the external magnet 23—Dominguez tandem 50—gallbladder towards the flank and caudal zone to expose the Calot triangle which contains the cystic artery and the cystic conduit surrounded by peritoneum. If necessary, more Dominguez tandem 50 can be placed for stronger traction force and exposure of the triangle. In this step, if the liver does not allow an easy dissection of the peritoneum and of the elements of the triangle, help can be achieved with the William retractor 47 (see FIG. 15) or with a string carrier needle (“needle of surgery without track”) placed at right hypochondrium. Then, the dissection of cystic conduit and the cystic artery is started, using instruments entering via the working canal, and being able of making ligatures with extracorporeal knots, placing the “hem-o-lock” clips or titanium LT200 clips, bipolar energy or an harmonic scalpel.

Cholangiography: Once identified the elements of the Calot triangle, the cystic conduit, is repaired by means of an external ligature approaching a non tight knot; the cysticotomy (eyelet in the conduit) is performed with scissors and it is catheterized via the working canal by using Valentina cannula 39 (see FIG. 14) fastening the catheter through hole of Valentina cannula 39 avoiding in this way contrast fluid Triyoson® reflux and also catheter displacement is avoided. Another option is inserting the catheter in percutaneous way, holding it within the cystic zone with prehensile clamp via the canal or, also, if a string pulling needle is used, it is possible to catheterize through this passage and anchor it within the cystic zone by prehensile clamp via the canal. After performing the biliary tract test (cholangiography) the catheter is removed and knot is fastened with the Valentina cannula 39. If necessary, the Hem-o-lok type clip or clips are placed to seal the conduit closing before being eventually cut. Arteries are sealed with a clip and further sectioned. In the event of finding calculus in the biliary tract, it is possible to try to move them to the duodenum by placing a spherical magnet in the choledochum with the Camila pliers 34, and then by means of the bell and magnet device 32 plus a needle 31, to slide the spherical magnet, thereby dragging the calculus.

4—Cholecystectomy: The gallbladder is detached from the liver. The necessary traction for the detachment of the gallbladder from the hepatic bed is achieved with external magnet 23 movements by the assistant. Coagulation and cut is performed as usual by electro-scalpel but from the working canal.

5—Extraction of Gallbladder: crocodile type clips 1 are released with Thomas pliers 13 at the same time the same Thomas pliers 13 takes the cystic remaining on the gallbladder side and it is removed under direct sight through the trocar, after it is released from magnetic field of external magnet 23 Dominguez tandem 50. Another option according to the case and/or preference of the surgeon is to insert a sterile bag that is positioned with one of crocodile type clips 1 (the one of infundibulum) and Thomas pliers 13 via the canal, for the introduction of the gallbladder in the bag and extraction of the piece protected to avoid umbilical infection.

6-Wash aspiration and control of haemostasis, removal of Dominguez tandem 50, evacuation of CO₂ by umbilical trocar and closing by planes, this step being done in the usual way.

Surgeries of Hiatus for Pathologies Like Achalasia, Hiatus Hernia and Esophagic Diverticulum

The surgery of hiatus and Achalasia are non-resective surgeries, that is to say, they do not remove organs, in the case of diverticulum, it is only resective for removing the diverticulum. For hiatus hernia treatment, which is an hernia, generally happening because of displacement of the stomach towards the thorax through the pillars of the diaphragm, the liver must be separated in order to expose these organs. For this, a William' retractor 47 is used, in order to separate the left hepatic lobe. The main objects of the surgery are to close the pillars that are the space through which the esophagus passes. The esophagus normally passes 1-2 cm away from the thorax to the abdominal cavity through diaphragm between the pillars of the diaphragm. By closing the pillars, if there is an hernia, the elevation of the stomach towards the thorax should be avoided. After this step, a funduplicature is performed to avoid the elevation of the stomach to the thorax and to avoid cholhydric acid reflux from stomach towards the esophagus by weakening of the lower esophagic sphincter. The sphincter is an non-voluntary moving muscle anatomically located at the esophagus-gastric union, and is part of the wall of these organs and, under normal pressure and functioning, it should avoid the acid reflux. In order to close the pillars it is usually enough to reduce the hernia and the settling of points between the pillars. The funduplicature is performed after dissection and closing of pillars, the passage of gastric fundus 360 degrees over its axis behind the esophagus (Nissen operation with the stomach the esophagus is surrounded) etc.

If necessary, in this step, to give more mobility to the stomach the short vessels running from stomach to spleen are sectioned, with a due haemostatic control. Stitches among the stomach-esophagus-stomach are made given to create a valve, which is calibrated with a probe within the gap of the esophagus-stomach that is introduced through the mouth.

With an hepatic Williams' retractor it is possible to expose the hiatus area and the dissection of the pillars of the diaphragm in usual way is begun, dissecting the esophagus and the esophagus-gastric union, in case of hernia, reduction of the same one, in case of Achalasia this step is not necessary and Heller miotomy is performed (a cut of 2 of the three layers of the esophagus-stomach, the serosa layer and muscular layer up to esophagic mucosa) according to technique with Electro scalpel.

In case of diverticulum, once it is detected, a magnet is placed inside its gap by means of endoscopy and with a Dominguez tandem it is moved for its dissection from outside the esophagic wall. Once the exposed, the diverticulum is resected and the esophagus is closed with sutures finishing the procedure with this step.

In this surgery probes with spherical magnets at their ends are used. These enter through the patient's mouth to the stomach being inserted by the anesthesiologist. Besides calibrating the funduplicature, in order to help to moving the stomach during the procedure, a Dominguez tandem 50 is used, entering by umbilical trocar, managing to move the esophagus and the stomach for the dissection of the pillars and hernia. With one or more Dominguez tandems and one external magnet, organs are positioned and are pulled to carry out the described steps and these are carried out with the aid of instruments that enter via the optics devices. In the case of Achalasia the technique is completed by stitching the stomach to the esophagic wall according to Dor technique (Heller-Dor' operation).

In difficult cases, usual laparoscopic instruments or 2-3 mm with more trocars can be used. Having ended the procedure, the separator and the tandem are removed, CO₂ is vented and the umbilical wound is closed.

Spleen Surgery (Splenectomy)

For splenectomy or spleen removal, spleen ligaments should be released. Once an umbilical trocar is placed, the section of the ligaments connecting and anchor the spleen begins. This is achieved by placing a Dominguez tandem 50 in the ligament to be sectioned. The ligament becomes tense with the aid of the external magnet, cutting is done on the ligament between spleen and the tandem using a pliers which enters via umbilical trocar. The same procedure is carried out with all ligaments. The William retractor-separator 47 is for separating the left hepatic lobe in case this lobe does not allow the sight of spleen or ligaments. The same retractor can also be used for better counter-traction of spleen in releasing the ligaments (in this case, the one contacting the separator probe is the spleen). In the event the colon, the intestine or the stomach must be moved, for obtaining better exposure of the surgical field, this can be done with Dominguez tandem 50 plus an external magnet (the clip which is used in these cases has no teeth, or the teeth of the clip are protected with a plastic covering that can be made from a cut-out of a serum guide).

The releasing of the pedicle or of the splenic thread, through which the arteries and vessels enter the spleen, is performed under haemostatic control with instruments inserted by the optical canal. After this step, the spleen is completely released; it must be eventually removed, and this can be done by placing it in a protective bag as in the gallbladder case. The surgery uses a surgical probe containing spherical magnets at the end entering via the stomach entrance and is inserted by the anesthetist. It is useful to move in this case the stomach with a Dominguez tandem 50 which enters by umbilical trocar for handling of splenic hilium. Finally, spleen is removed within the bag, thorough the navel. If necessary (because of size) it is morcelled within the bag and it is removed through navel. Tandem and retractor are removed, haemostasia is controlled, CO₂ is vented and navel is closed.

Appendix and Colon Surgery

1—One or more Dominguez tandem 50 are used, plus an external magnet 23 for handling of mesoappendix-mesocolon or nontraumatic, clamp-type, clip for tandem in colon wall.

2—Probes of several diameters with magnets for colon which are inserted by the anus.

Gynecological Surgery

1—One or more Dominguez tandems 50 for ovaries, tubes, uterus, infundibulum, etc. plus an external magnet 23.

2—For mobilizing or anchoring the uterus, it is used a William Retractor 47 or an intrauterine magnet (placed by hysteroscopy) with Dominguez tandem 50, placed via umbilical trocar which is anchored to the uterus. Both, the intrauterine magnet and Dominguez tandem are handled with an external magnet 23.

Surgery Abdominal Wall Surgery:

1—One or more Dominguez tandems 50 plus external magnets 23 are used for handling the peritoneum and Tap technique meshes (Transperineal), with trocar for inguinal and crural hernias. The same are used for hernias.

2—Curved and straight steel needles with 150 cm long threads, measuring 0.30-0.35-0.40 cm in diameter for extracorporeal knots and continuous or separated sutures.

Urolologic Surgery

1—Spherical Magnet for uretral lithos.

2—One or more Dominguez tandems 50 are used, plus External magnet 23 for kidney.

3—William retractor 47 is used for separating the liver.

4—A Probe 23 with small magnet at the end for positioning inside the urether, using cystoscopy for location, identification and handling it using an intra-abdominal magnet.

1. A magnetic surgical device for manipulating tissue during surgery comprising:
a grip that allows firmly taking hold of tissue;
a magnetic system;
means of interconnection between said grip and said magnetic system; and
a ferromagnetic steel tube that houses the magnetic system and provides a high magnetic-permeability material that improves the magnetic field polarization.

2. The magnetic surgical device of claim 1, wherein said grip and said means of interconnection are made of non-ferromagnetic material.

3. The magnetic surgical device of claim 1, wherein said ferromagnetic steel tube comprises a first cavity for housing the magnetic system and another second cavity for housing the end of the interconnection means.

4. The magnetic surgical device of claim 1, wherein a safety string is anchored to the second cavity of the steel tube, the safety string providing a link that allows recovering the tube when it accidentally falls during a surgery intervention, and also placing and removing it from a patient.

5. The magnetic surgical device of claim 4, wherein the magnetic system is formed by one or more annular magnets, the safety string crossing trough the centre of the annular magnets to come out the surgical device.

6. The magnetic surgical device of claim 4, wherein the steel tube comprises a side hole next to the header, the safety string crossing trough said side hole to come out the surgical device.

7. The magnetic surgical device of claim 1, wherein said grip consists of a crocodile type clip comprising:
two grips linked to each other, wherein said grips have a sawtooth profile on the side on which they contact each other when the clip is closed, and wherein said grips have fins on the side on which they are not in contact to each other when the clip is closed;
a pin linking said grips and allowing these to be articulated one with the other; and
a spring that keeps said grips in contact in the closed position of the clip.

8. The magnetic surgical device of claim 1, wherein said annular magnets are made of a rare earth material and they are gold, nickel, silver or titanium.

9. A surgical apparatus for positioning an external magnet during surgery comprising:
a cup-shaped device upwardly closed by a tap that has a central threaded through-hole and downwardly open;
a central pin threaded to said upper tap of the cup-shaped device;
a manually driven wheel rigidly fixed to said central pin; and
a cylindrical magnet housed within the cup-shaped device and rigidly fixed to said central pin;
wherein upon manually turning said driven wheel and said central pin, the cylindrical magnet within the cup-shaped device is vertically moved allowing regulating the strength of the magnetic field.

10. The surgical apparatus of claim 9, wherein said cylindrical magnet housed within the cup-shaped device is permanent magnet made of a rare earth material.

11. A surgical device for manipulating tissue during surgery comprising:
a clip that allows firmly taking hold of tissue;
a needle; and
a string that interconnect said clip and said needle.

12. The surgical device of claim 11, wherein the clip is made of a non-ferromagnetic material.

13. The surgical device of claim 11, wherein the clip is made of a ferromagnetic material.
